(19) United States
(12) Patent Application Publication
(10) Pub. No.: US 2024/0300914 A1
(43) Pub. Date: Sep. 12, 2024

(54) KC2-Type Lipids

(71) Applicant: NanoVation Therapeutics Inc., Vancouver (CA)

(72) Inventors: Marco A. Ciufolini, Vancouver (CA); Fariba Saadati, Vancouver (CA); Anthony C. Y. Tam, Vancouver (CA); Daniel Kurek, Vancouver (CA); Dominik Witzigmann, Binzen (DE); Jayesh Kulkarni, Vancouver (CA)

(73) Assignee: NanoVation Therapeutics Inc., Vancouver, BC (CA)

(21) Appl. No.: 18/564,769

(22) PCT Filed: May 26, 2022
(86) PCT No.: PCT/CA2022/050853

§ 371 Date: Nov. 28, 2023

Related U.S. Application Data
(60) Provisional application No. 63/194,471, filed on May 28, 2021.
(60) Provisional application No. 63/214,977, filed on Jun. 25, 2021.

(51) Int. Cl. C07D 317/28; A61K 9/51; C07C 45/67; C07C 67/343; C07D 317/20; C07D 317/24

(52) U.S. Cl. CPC C07D 317/28; A61K 9/5123; C07C 45/676; C07C 67/343; C07D 317/20; C07D 317/24

(57) ABSTRACT

Provided is lipid and methods of making such lipid having the structure of Formula A:

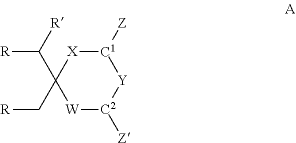

each R is an alkyl having a carbon backbone of C₁₂ to C₁₆ having 1 to 3 C═C double bonds, at least one having Z-geometry;
R′ is an optional alkyl of C₂ to C₂₄ having 0 to 3 C═C double bonds;
each R, and the R′ alkyl group if present, is optionally substituted at one or more positions with a C₁ to C₃ alkyl;
W and X are O or S;
Y is absent and C¹ and C² are bonded, or
Y is present and is a metheno, etheno or ethyno bridge optionally substituted with an alkylamino chain [—(CH₂)ₘ—NG¹G²G³],
wherein m is 1-5 and G¹, G² are, independently, C₁ to C₃ alkyl, G³ is absent, hydrogen or C₁ to C₃ alkyl,
or wherein NG¹G²G³ is a heterocycle; and
Z and Z′ are H or alkylamino.

### TECHNICAL FIELD

Provided herein are lipids that may be formulated in a delivery vehicle so as to facilitate the encapsulation of a wide range of therapeutic agents or prodrugs therein, such as, without limitation, nucleic acids (e.g., RNA or DNA), proteins, peptides, pharmaceutical drugs and salts thereof.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of lipid nanoparticles (LNPs) is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the lipid nanoparticles are taken up by a cell by endocytosis, the ability of these lipids to ionize at low pH enables endosomal escape. This in turn enables the nucleic acid to be released into the intracellular compartment. While most research on cationic LNPs has focused on the formulation of nucleic acid, the delivery of other therapeutic agents or prodrugs besides nucleic acid is possible as well using the delivery platform.

Significant research has been devoted to identifying amino lipids with high potency. An ionizable lipid, referred to as DLin-MC3-DMA or “MC3” (dilinoleyl-methyl-4-dimethylaminobutyrate), constitutes the state-of-the-art ionizable lipid for siRNA formulations. This ionizable lipid is a key component of Onpattro®, a lipid nanoparticle formulation incorporating siRNA that silences genes causing a genetic neurodegenerative disease referred to as hereditary transthyretin-mediated amyloidosis. Such formulations containing MC3 constituted the first small interfering RNA (siRNA) based treatments to be approved by the U.S. Food and Drug Administration (FDA).

The MC3 ionizable lipid is widely regarded as being an improved version of another amino lipid referred to as KC2, being about 3 times more efficacious. In a study of over 50 amino lipids, MC3 was identified as having an ED₅₀ or 0.03 while that of KC2 was 0.10 for FVII gene silencing in mice using siRNA. (Jayaraman et al., 2012, Angew. Chem. Int. Ed., 51:8529-8533). This means that formulations containing MC3 require about 3 times less siRNA to attain the same end-result as similar formulations based on KC2. Since nucleic acid is costly, this translates into considerable savings for large scale manufacture of the ionizable lipid.

Both MC3 and KC2 amino lipids (structures below) have two carbon chains (denoted as “R” herein) converging onto a single carbon atom, which in turn serves as the anchoring point for an ionizable terminal “head” group.

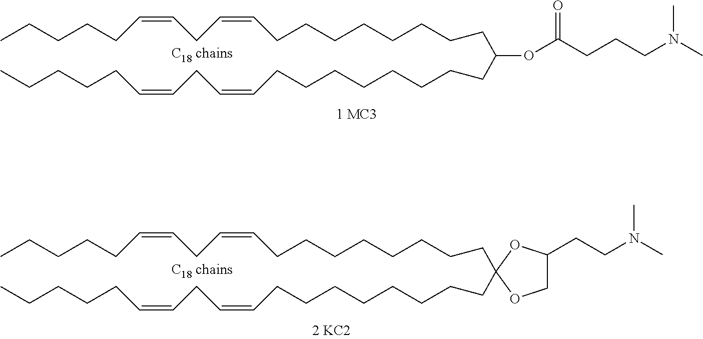

In both MC3 and KC2, the carbon chains are unsaturated C18 moieties derived from linoleic acid or a corresponding ester. These 18 carbon unsaturated chains are the best chains yet identified for maximum efficacy of siRNA formulations. (Semple et al., 2010, Nat. Biotechnol. 28:172-176 and Heyes et al., 2005, J. Controlled Release, 107:276-287). Further, amino lipids with chains shorter than 18 carbon atoms are difficult to prepare using conventional synthetic routes. While shorter-chain lipids could be produced from esters of unsaturated fatty acids incorporating fewer than 18 C atoms, such esters are either not found in nature, or are extremely costly to synthesize using known methods. Accordingly, little attention has been devoted to examining amino lipids for nucleic acid delivery having unsaturated chains of fewer than 18 carbon atoms. There is a need in the art for ionizable lipids for delivery of therapeutic agents or prodrugs that have a potency that is improved or comparable to known lipids and that can be manufactured conveniently and cost effectively.

### Definitions

As used herein, a “KC2-type lipid” refers to any lipid, including but not limited to an ionizable lipid, having a structure as defined by Formula A herein or equivalents thereof.

As used herein, the term “ionizable lipid” refers to a lipid that, at a given pH, is in an electrostatically neutral form and that may either accept or donate protons, thereby becoming electrostatically charged, and in which the electrostatically neutral form has a calculated logarithm of the partition coefficient between water and 1-octanol (i.e., a c Log P) that is greater than 8.

As used herein, the term “alkyl” with reference to an R group as described herein is a carbon-containing chain that is linear or branched and that has varying degrees of unsaturation.

As used herein, the term “C₁ to C₃ alkyl” refers to a linear or branched carbon chain having a total of up to 3 carbon atoms, optionally unsaturated. As used herein, the term “helper lipid” means a compound selected from: a sterol such as cholesterol or a derivative thereof; a diacylglycerol or a derivative thereof, such as a glycerophospholipid, including phosphatidic acid (phosphatidate) (PA), phosphatidylethanolamine (cephalin) (PE), phosphatidylcholine (PC), phosphatidylserine (PS), and the like; and a sphingolipid, such as a ceramide, a sphingomyelin, a cerebroside, a ganglioside, or reduced analogues thereof, that lack a double bond in the sphingosine unit. The term encompasses lipids that are either naturally-occurring or synthetic.

As used herein, the term “delivery vehicle” includes any preparation in which the lipid described herein is capable of being formulated and includes but is not limited to delivery vehicles comprising helper lipids.

As used herein, the term “nanoparticle” is any suitable particle in which the lipid can be formulated and that may comprise one or more helper lipid components. The one or more lipid components may include an ionizable lipid prepared by the method described herein and/or may include additional lipid components, such as a helper lipid. The term includes, but is not limited to, vesicles with one or more bilayers, including multilamellar vesicles, unilamellar vesicles and vesicles with an electron-dense core. The term also includes polymer-lipid hybrids, including particles in which the lipid is attached to a polymer.

As used herein, the term “encapsulation,” with reference to incorporating a cargo molecule within a delivery vehicle refers to any association of the cargo with any component or compartment of the delivery vehicle such as a nanoparticle.

### SUMMARY

The disclosure seeks to address one or more limitations of known art or to provide useful alternatives thereof.

The present disclosure is based, at least in part, on the surprising discovery that certain KC2-type amino lipids having unsaturated chains with less than 18 carbon atoms have enhanced nucleic acid delivery efficacy when formulated in a delivery vehicle. The potency of such short-chain amino lipids is significantly better or comparable to that of the state-of-the-art amino lipid, MC3. For example, the inventors have found that a short-chain KC2-type lipid (e.g., having unsaturated C₁₇ moieties) has an mRNA efficacy in vitro and in vivo when formulated in a delivery vehicle that is far superior to that of the longer chain, benchmark lipid MC3 (having unsaturated Cis moieties derived from linoleic acid). The inventors have further found that siRNA-containing delivery vehicles including short-chain KC2-type lipids (e.g., having unsaturated C17 moieties) have comparable potency to MC3, which is contrary to studies showing that KC2 is three times less efficacious than MC3 and that the dilinoleyl chain (unsaturated Cis) is considered optimal for activity.

Further, the inventors have identified a class of short chain (less than Cis unsaturated moieties), KC2-type lipids that are easily prepared by a method described herein in which longer chain fatty acid esters (e.g., commercially available unsaturated C18 to C₂₂) are shortened and subsequently converted to the KC2-type lipids in a synthesis route including a Claisen condensation step as described herein. Thus, the present disclosure further addresses previous shortcomings with synthesizing KC2-type lipids having less than 18 carbon atoms.

Other objects, features, and advantages of the present disclosure will be apparent to those of skill in the art from the following detailed description and figures.

### BRIEF DESCRIPTION OF THE DRAWINGS

FIG. 1A is a bar graph showing entrapment (%), particle size and polydispersity index (PDI) of mRNA-containing lipid nanoparticles (LNPs) comprising the ionizable lipid nor-KC2 or DLin-MC3-DMA (MC3). The LNPs are composed of 50/10/38.5/1.5 mol % of ionizable lipid/DSPC/chol/PEG-DMG and the amine-to-phosphate (N/P) was 6.

FIG. 1B shows luminescence intensity as a function of mRNA concentration (log scale) after addition of the mRNA-containing LNPs comprising the ionizable lipid nor-KC2 or MC3 to HuH7 cells. The LNPs are composed of 50/10/38.5/1.5 mol % of ionizable lipid/DSPC/chol/PEG-DMG (N/P=6) and the mRNA encodes firefly luciferase.

FIG. 1C shows luminescence intensity/mg in the liver (left graph) or spleen (right graph) for the mRNA-containing LNPs comprising the ionizable lipid nor-KC2 or MC3 after 4 hours post-intravenous administration to C57Bl/6J mice. The LNPs contain 50/10/38.5/1.5 mol % of ionizable lipid/DSPC/chol/PEG-DMG (N/P=6).

FIG. 2A is a bar graph showing entrapment (%), particle size and PDI of siRNA-containing LNPs comprising the ionizable lipid nor-KC2 or MC3. The LNPs contain 50/10/38.5/1.5 mol % of ionizable lipid/DSPC/chol/PEG-DMG and the N/P was 3.

FIG. 2B shows normalized luminescence intensity (%) after addition of siRNA-containing LNPs comprising the ionizable lipid nor-KC2 or MC3 to 22Rv1 cells modified to stably express luciferase. The LNPs contain 50/10/38.5/1.5 mol % of ionizable lipid/DSPC/chol/PEG-DMG (N/P=3) and the siRNA encodes luciferase.

### DETAILED DESCRIPTION

KC2-Type Lipids

The present disclosure provides a KC2-type lipid having the structure of Formula A:

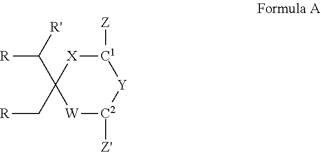

each R is independently an alkyl group having a carbon backbone of C₁₂ to C₁₆, each alkyl group having 1 to 3 C═C double bonds, or 1 to 2 double bonds or 2 double bonds, wherein at least one of the double bonds is of Z geometry, and most advantageously each double bond of R is of Z geometry;
R′ is an optional alkyl group having a backbone of C₂ to C₂₄ and having 0 to 3 C═C double bonds;
each R, and the R′ alkyl group if present, is optionally substituted at one or more positions with a C₁ to C₃ alkyl group;
W and X are independently O or S, or most advantageously both oxygen;
C¹ and C² are carbon atoms;
Y is either absent or present,
wherein if Y is absent, then C1 and C² are directly linked by a C—C bond, and
when Y is present, Y is a bridge selected from a metheno (CH₂) bridge, etheno (CH₂CH₂) and ethyno (CH═CH) bridge, the bridge optionally substituted with an alkylamino chain as defined by [—(CH₂)ₘ—NG¹G²G³],
wherein m is 1-5 or 1-3 and G¹ and G² are, independently, the C₁ to C₃ alkyl group, such as a methyl, G³ is absent (i.e., it is a lone pair), a hydrogen or a C₁ to C₃ alkyl group, such as a methyl, or wherein NG¹G²G³ is a nitrogen heterocycle; and
Z and Z′ are, independently, H, or the alkylamino chain as defined by [(CH₂)ₘ—NG¹G²G³].

In one embodiment, each R alkyl group has two double bonds of Z geometry.

In one embodiment, the C₁ to C₃ alkyl group of Formula A that is substituted on the R or R′ alkyl group is selected from methyl, ethyl, propyl, isopropyl, butyl, isobutyl and tert-butyl. The C₁ to C₃ alkyl group typically replaces a hydrogen atom on the R and/or R′ alkyl group carbon backbone.

In another embodiment, the lipid of Formula A has the structure of Formula B below.

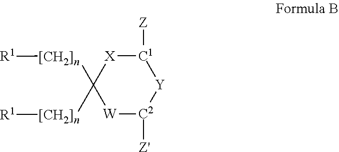

wherein the R alkyl group (see Formula A) is represented by an R¹—[CH₂]ₙ moiety, which is a linear alkyl group that is C₁₂ to C₁₇, wherein the n of the [CH₂]ₙ moiety is 2-7, wherein each R¹ is C₁₅ or less and the R¹—[CH₂]ₙ moiety is optionally substituted with the C₁ to C₃ alkyl group; and
wherein each R¹ independently has 1-3 double bonds, or 1-2 double bonds or 2 double bonds, wherein at least one of the double bonds is of Z geometry, and most advantageously each double bond of R¹ is of Z geometry.

In another embodiment, the cyclic moiety to which the R alkyl groups of Formula B are each attached does not contain a Y atom (i.e., C¹ and C² are directly linked) and the lipid has the structure of Formula C as set out below.

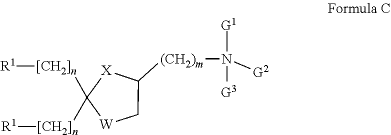

wherein m is 1-5 or 1-3;
G¹ and G² are, independently, a C¹ to C³ alkyl, most advantageously each G¹ and G² is a methyl group; and
G³ is absent (i.e., it is a lone pair) or a hydrogen (as dependent on pH).

In another embodiment, the lipid of Formula B has the structure of Formula D set forth below.

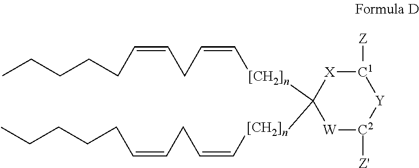

wherein n is 2 to 7.

In another non-limiting example, the lipid of general Formula B above has the structure of Formula E provided below.

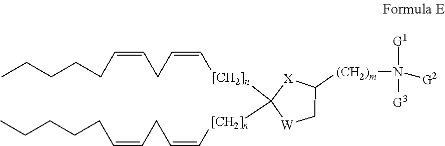

wherein n is 2 to 7.

In yet a further embodiment, the lipid of Formula B has the structure of Formula F below:

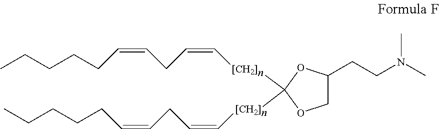

wherein n is 2 to 7.

In one embodiment, the lipid has the structure of nor-KC2:

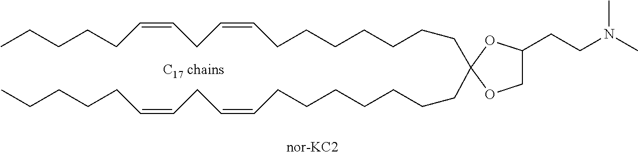 [SMILES: CCCCC/C=C\C/C=C\CCCCCCCC1(CCCCCCC/C=C\C/C=C\CCCCC)OCC(CCN(C)C)O1]

### Methods to Produce KC2-Type Lipids Having C₇ or Shorter R Alkyl Groups

KC2-type lipids having C₁₇ or shorter R alkyl groups can be prepared using the methods described below. While a linoleate ester (e.g., methyl linoleate) is the starting material in the synthetic schemes described below, as would be appreciated by those of skill in the art, other fatty acids could serve as the starting material and the schemes set forth below are merely illustrative of select embodiments.

As previously noted, in both KC2 and MC3 ionizable amino lipids, the carbon chains are unsaturated C₁₈ moieties derived from linoleic acid or a corresponding ester.

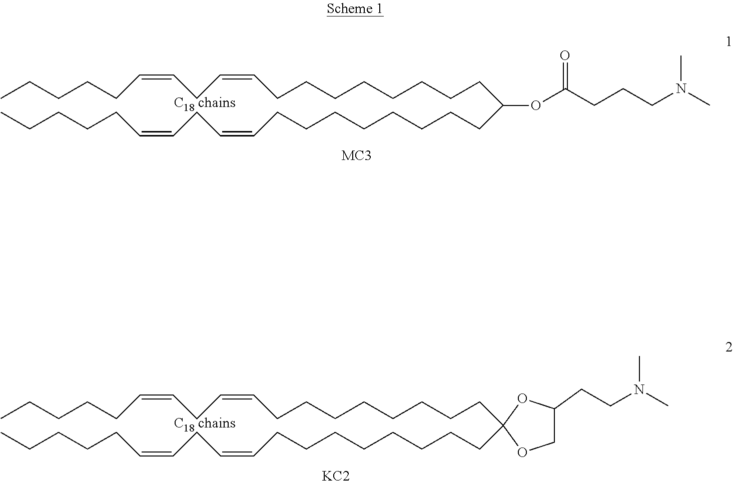

KC2 2 and analogues thereof may be represented by general formula 3 (Scheme 2 below). The methods described herein provide for the synthesis of KC2-type lipids 4

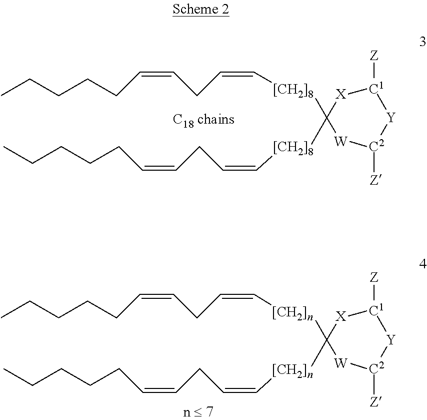

wherein n is 2-7;
W and X are independently O or S;
C¹ and C² are carbon atoms;
Y is absent and C¹ and C² are directly linked by a C—C bond, or
Y is present and is a bridge selected from a metheno (CH₂) bridge, etheno (CH₂CH₂) and ethyno (CH═CH) bridge, the bridge optionally substituted with an alkylamino chain as defined by [—(CH₂)ₘ—NG¹G²G³],
wherein m is 1-5 and G¹ and G² are, independently, a C₁ to C₃ alkyl, G³ is absent (i.e., it is a lone pair), a hydrogen or a C₁ to C₃ alkyl,
or wherein NG¹G²G³ is a nitrogen heterocycle; and
Z and Z′ are, independently, H, or the alkylamino chain as defined by [(CH₂)ₘ—NG¹G²G³].

Lipids of the type 4 wherein n=7 are described in co-pending and co-owned U.S. Provisional Application No. 63/194,471 titled “Method for Producing an Ionizable Lipid”, which is incorporated herein by reference.

U.S. Provisional Application No. 63/194,471 also describes methods to make ionizable lipids from a generic fatty acid ester 5 (Scheme 3). As described therein, the fatty acid ester is subjected to Claisen condensation under Mukaiyama conditions, resulting in ketoester 6, which may optionally undergo addition of an R² alkyl group.

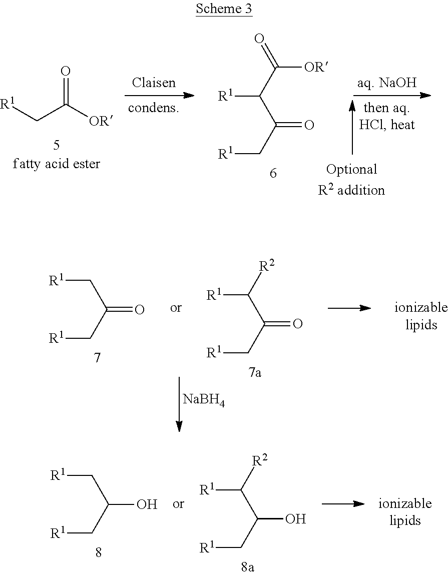

The ketoester is subsequently hydrolyzed and decarboxylated to provide ketones 7 (or 7a), which may be further reduced to alcohols 8 (or 8a). Ketalization of ketones 7 yields KC2-type lipids and esterification of alcohols 8 yields MC3-type lipids as set forth in co-owned and co-pending U.S. Provisional Application No. 63/194,471.

The chemistry is such that an ester incorporating n carbon atoms in its acid portion will provide ionizable lipids with hydrophobic chains having n-1 C atoms. For example, methyl linoleate, a C₁₈ fatty acid ester, yields nor-KC2 displaying C₁₇ chains (Scheme 4). The structures of such nor-lipid can be represented as structure 4 above, in which n=7.

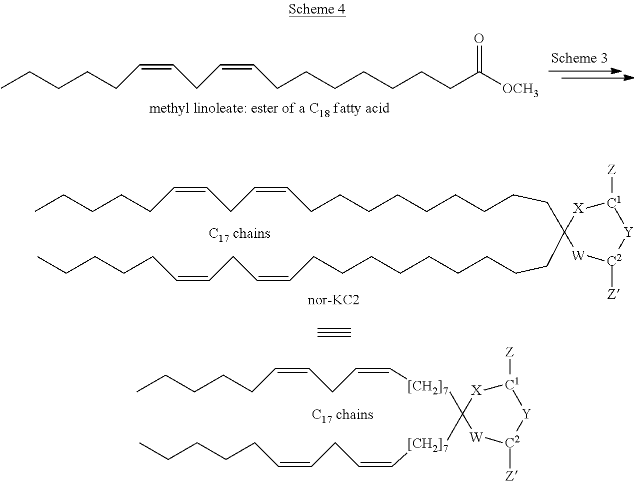

Shorter-chain lipids of the type 4 would be available from esters of unsaturated fatty acids incorporating fewer than 18 carbon atoms.

### Formulation of the Lipid in a Delivery Vehicle

The KC2-type lipid of the disclosure may be formulated in a variety of drug delivery vehicles (also referred to herein as a “delivery vehicle”) known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, the KC2-type lipid of the disclosure is formulated in a delivery vehicle by mixing them with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof. These lipids may be synthesized or obtained from natural sources, such as from egg.

A suitable ceramide derivative is egg sphingomyelin or dihydrosphingomyelin.

Delivery vehicles incorporating the KC2-type lipids of the disclosure can be prepared using a wide variety of well-described formulation methodologies known to those of skill in the art, including but not limited to extrusion, ethanol injection and in-line mixing. In one embodiment, the preparation method is an in-line mixing technique in which aqueous and organic solutions are mixed using a rapid-mixing device as described in Kulkarni et al., 2018, ACS Nano, 12:4787 and Kulkarni et al., 2017, Nanoscale, 36:133347, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid.

Nanoparticles comprising the KC2-type lipid of the disclosure may alternatively be prepared from polymers without lipids. Such nanoparticles may comprise a concentrated core of a therapeutic agent that is surrounded by a polymeric shell or may have a solid or a liquid dispersed throughout a polymer matrix.

The KC2-type lipids described herein can also be incorporated into emulsions, which are drug delivery vehicles that contain oil droplets or an oil core. An emulsion can be lipid-stabilized.

For example, an emulsion may comprise an oil filled core stabilized by an emulsifying component such as a monolayer or bilayer of lipids.

The KC2-type lipid may be incorporated into a micelle. Micelles are self-assembling particles composed of amphipathic lipids or polymeric components that are utilized for the delivery of agents present in the hydrophobic core.

A further class of drug delivery vehicles known to those of skill in the art that can be used to formulate the KC2-type lipid herein is a carbon nanotube.

### Delivery of Nucleic Acid, Genetic Material, Proteins, Peptides or Other Charged Agents

The KC2-type lipid disclosed herein may facilitate the incorporation of a compound or molecule (referred to herein also as “cargo” or “cargo molecule”) bearing a net negative or positive charge into the delivery vehicle and subsequent delivery to a target cell in vitro or in vivo.

In one embodiment, the molecule is genetic material, such as a nucleic acid. The nucleic acid includes, without limitation, RNA, including small interfering RNA (siRNA), small nuclear RNA (snRNA), micro RNA (miRNA), messenger RNA (mRNA) or DNA such as vector DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one embodiment, the cargo is an mRNA, which includes a polynucleotide that encodes at least one peptide, polypeptide or protein. The mRNA includes, but is not limited to, small activating RNA (saRNA) and trans-amplifying RNA (taRNA), as described in co-pending U.S. provisional Application No. 63/195,269, titled “mRNA Delivery Using Lipid Nanoparticles”, which is incorporated herein by reference.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is a chemically synthesized molecule, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to encapsulate mRNAs of a variety of lengths. In some embodiments, the present disclosure may be used to encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on the 5′ end, and a “tail” on the 3′ end. The presence of the cap is important in providing resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences may be incorporated into mRNA for in vitro studies or for in vivo studies in animal models to assess biodistribution.

In another embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

The siRNA encompassed by embodiments of the disclosure may be used to specifically inhibit expression of a wide variety of target polynucleotides. The siRNA molecules targeting specific polynucleotides may be readily prepared according to procedures known in the art. An siRNA target site may be selected, and corresponding siRNAs may be chemically synthesized, created by in vitro transcription, or expressed from a vector or PCR product. A wide variety of different siRNA molecules may be used to target a specific gene or transcript. The siRNA may be double-stranded RNA, or a hybrid molecule comprising both RNA and DNA, e.g., one RNA strand and one DNA strand. The siRNA may be of a variety of lengths, such as 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA. Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic importance in diseases that are an outcome of splicing defects.

In another embodiment, the cargo is a DNA vector as described in co-owned and co-pending U.S. Serial No. U.S. Application No. 63/202,210 titled “DNA Vector Delivery Using Lipid Nanoparticles”, which is incorporated herein by reference. The DNA vectors may be administered to a subject for the purpose of repairing, enhancing or blocking or reducing the expression of a cellular protein or peptide. Accordingly, the nucleotide polymers can be nucleotide sequences including genomic DNA, cDNA, or RNA.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will preferably have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16(11):1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present method can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the DNA vector is a nanoplasmid or a minicircle.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the lipid described herein. Optionally, a ribonucleoprotein complex may be incorporated into a delivery vehicle comprising the lipid described herein. Likewise, the disclosure includes embodiments in which genetic material encoding DNA binding and cleavage domains of a zinc finger nuclease or TALEN system are incorporated into a delivery vehicle together with the KC2-type lipid of the disclosure.

While a variety of nucleic acid cargo molecules are described above, it will be understood that the above examples are non-limiting and the disclosure is not to be considered limiting with respect to the particular cargo molecule encapsulated in the delivery vehicle.

For example, the KC2-type lipid described herein may also facilitate the incorporation of proteins and peptides into a delivery vehicle, which includes ribonucleoproteins. This includes both linear and non-linear peptides, proteins or ribonucleoproteins.

While pharmaceutical compositions are described above, the KC2-type lipid described herein can be a component of any nutritional, cosmetic, cleaning or foodstuff product.

### Pharmaceutical Formulations

In some embodiments, the delivery vehicle comprising the cargo molecule is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In one embodiment, the pharmaceutical compositions is administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The pharmaceutical composition comprises pharmaceutically acceptable salts and/or excipients.

The compositions described herein may be administered to a patient. The term patient as used herein includes a human or a non-human subject.

The following examples are given for the purpose of illustration only and not by way of limitation on the scope of the invention.

### EXAMPLES

### Materials

The lipid 1,2-distearoyl-sn-glycero-3-phosphorylcholine (DSPC) and 1,2-dimyristoyl-rac-glycero-3-methoxypolyethylene glycol-2000 (PEG-DMG) were purchased from Avanti Polar Lipids (Alabaster, AL). Cholesterol and 10× Phosphate Buffered Saline (pH 7.4) were purchased from Sigma Aldrich (St Louis, MO). The ionizable amino-lipid was synthesized as previously described in U.S. Provisional Application No. 63/194,471 titled “Method for Producing an Ionizable Lipid”, which is incorporated herein by reference.

An mRNA encoding firefly luciferase purchased from APExBIO Technology LLC (Houston, TX) was used to analyse luciferase activity.

An siRNA targeted against firefly luciferase purchased from Integrated DNA Technologies (IDT, Coralville, IA) was used to assess ability of LNP to knockdown firefly luciferase in a cell line.

### Methods

Preparation of Lipid Nanoparticles (LNP) Containing mRNA or siRNA

Lipids used in the formulation, nor-KC2 or MC3, DSPC, cholesterol, and PEG-DMG, were dissolved in ethanol at the appropriate ratios to a final concentration of 10 mM total lipid. Nucleic acid (siRNA or mRNA) was dissolved in an appropriate buffer such as 25 mM sodium acetate pH 4 or sodium citrate pH 4 to a concentration necessary to achieve the appropriate amine-to-phosphate ratios. The aqueous and organic solutions were mixed using a rapid-mixing device as described in Kulkarni et al., 2018, ACS Nano, 12:4787 and Kulkarni et al., 2017, Nanoscale, 36:133347 (each incorporated herein by reference) at a flow rate ratio of 3:1 (v/v; respectively) and a total flow rate of 20 mL/min. The resultant mixture was dialyzed directly against 1000-fold volume of PBS pH 7.4. All formulations were concentrated using an Amicon centrifugal filter unit and analysed using the methods described below.

### Analysis of LNP

Particle size analysis of LNPs in PBS was carried out using backscatter measurements of dynamic light scattering with a Malvern Zetasizer (Worcestershire, UK). The reported particle sizes correspond to the number-weighted average diameters (nm). Total lipid concentrations were determined by extrapolation from the cholesterol content, which was measured using the Cholesterol E-Total Cholesterol Assay (Wako Diagnostics, Richmond, VA) as per the manufacturer's recommendations. Encapsulation efficiency of the formulations was determined using the Quant-iT RiboGreen Assay kit (Invitrogen, Waltham, MA). Briefly, the total siRNA or mRNA content in solution was measured by lysing lipid nanoparticles in a solution of TE containing 2% Trioton Tx-100, and free DNA vector in solution (external to LNP) was measured based on the RiboGreen fluorescence in a TE solution without Triton. Total siRNA or mRNA content in the formulation was determined using a modified Bligh-Dyer extraction procedure. Briefly, LNP formulations containing siRNA or mRNA were dissolved in a mixture of chloroform, methanol, and PBS that results in a single phase and the absorbance at 260 nm measured using a spectrophotometer.

### In Vitro Analysis in Huh7 Cells

Huh7 cells were cultured in Dulbecco's Modified Eagle Medium (DMEM) supplemented with 10% Fetal Bovine Serum (FBS). For cell treatments, 10,000 cells were added to each well in a 96-well plate. 24 hours later, the medium was aspirated and replaced with medium containing diluted LNP at the relevant concentration over a range of 0.03-10 μg/mL mRNA. Expression analysis was performed 24 hours later, and luciferase levels measured using the Steady-Glo Luciferase kit (Promega). Cells were lysed using the Glo Lysis buffer (Promega).

### In Vivo Analysis in C57Bl/6 Mice

LNP-mRNA encoding firefly luciferase were injected intravenously (tail-vein) into 6-8 wk old C57BL/6 mice. Four hours following injection, the animals were euthanized and the liver and spleen and isolated. Tissue was homogenized in Glo Lysis buffer and a luciferase assay performed using the Steady Glo Luciferase assay kit (as per manufacturers recommendations).

Organic Synthesis of nor-KC2

The synthesis of nor-KC2 from methyl linoleate was carried out as set forth below. As discussed, the synthesis of nor-KC2 involves subjecting the fatty acid ester to Claisen condensation under Mukaiyama conditions, resulting in ketoester 6 as described in Scheme 3 above. The ketoester 6 is subsequently hydrolyzed and decarboxylated to provide a ketone 7, which may be further reduced to an alcohols 8. Ketalization of ketone 7 yields KC2-type lipids as set forth in co-owned and co-pending U.S. Provisional Application No. 63/194,471.

Unless otherwise specified, all reagents and solvents were commercial products and were used without further purification, except THF (freshly distilled from Na/benzophenone under Ar), CH₂Cl₂ (freshly distilled from CaH₂ under Ar). “Dry methanol” was freshly distilled from magnesium turnings. All reactions were performed under an argon atmosphere. Reaction mixture from aqueous workups were dried by passing over a plug of anhydrous Na₂SO₄ held in a filter tube and rotary-evaporated under reduced pressure. Thin-layer chromatography was performed on silica gel plates coated with silica gel (Merck 60 F254 plates) and column chromatography was performed on 230-400 mesh silica gel. Visualization of the developed chromatogram was performed by staining with I₂ or potassium permanganate solution. Nuclear magnetic resonance spectra, ¹H (300 MHz) and ¹³C NMR (75 MHz), were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the 6 scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

### (a) Synthesis of ketoester from methyl linoleate: methyl (11Z,14Z)-2-((7Z,10Z)-hexadeca-7,10-dien-1-yl)-3-oxoicosa-11,14-dienoate

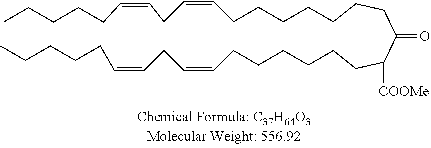 [SMILES: CCCCC/C=C\C/C=C\CCCCCCCC(=O)C(CCCCCC/C=C\C/C=C\CCCCC)C(=O)OC]

A solution of TiCl₄ (9.6 g, 5.7 mL, 45.0 mmol) in toluene (12 mL) was added dropwise to a cold (0° C., ice bath), stirred solution of a methyl linoleate (30.0 mmol) and tributylamine (Bu₃N) (10.2 g, 12.9 mL, 54.0 mmol) in toluene (50.0 mL). After stirring at 0° C. for 1.5 h, the reaction was complete as determined by TLC and ¹H NMR. The reaction solution was then diluted with hexanes (60 mL), and water (60 mL) was cautiously added. Addition of water caused evolution of heat, so the temperature of the mixture was controlled by thorough stirring and cooling in an ice bath. The organic phase was separated and the aqueous phase was extracted with more hexane (2×40 mL). The combined organic extracts were washed with water, passed over a plug of anhydrous Na₂SO₄ and concentrated under vacuum. Proton NMR analysis of the residue indicated the presence of some residual toluene. Suspended inorganic matter (likely TiO₂) may also be present. The crude product may be purified by column chromatography (3% diethyl ether in hexanes) to afford pure ketoester (96%) but may be advanced directly to the next step. NMR indicated that the product existed as a mixture of keto (major) and enol derivatives, typically in a 2:1 ratio. ¹H NMR (keto form) δ 5.37 (m, 8H), 3.77 (s, 3H), 3.45 (t, 1H), 2.79 (t, 4H), 2.40 (t, 2H), 2.07 (m, 8H), 1.85-1.20 (m, 32H), 0.91 (t, 6H). ¹³C NMR (keto form) δ 205.6, 170.6, 130.2, 130.1, 129.9, 129.8, 59.2, 52.4, 42.0, 32.1, 29.9, 29.8, 29.8, 29.7, 29.5, 29.4, 29.4, 29.3, 29.2, 29.1, 28.4, 27.6, 27.4, 27.3, 27.3, 23.6, 22.8, 14.3 (some peaks are doubled). LRMS: m/z 557 [M+H]+, 579 [M+Na]+

### (b) Hydrolysis and decarboxylation of the ketoester to produce the ketone: (6Z,9Z,26Z,29Z)-Pentatriaconta-6,9,26,29-tetraen-18-one

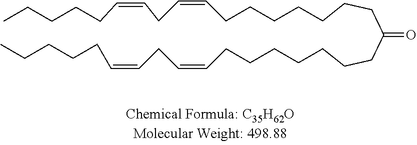 [SMILES: CCCCC/C=C\C/C=C\CCCCCCCC(=O)CCCCCCC/C=C\C/C=C\CCCCC]

Aqueous 10% w/vol NaOH (5 mL) was added to a solution of the above crude O ketoester (5.0 g) in 95% ethanol (25 mL). The mixture was stirred at room temperature overnight. The reaction was checked for completion by adding 3-4 drops of the reaction mixture to 3 N aqueous HCl solution (0.5 mL), extracting the mixture with hexanes, evaporating the combined extracts to dryness, and checking the residue by ¹H NMR. The disappearance of the OCH₃ signal and a downfield shift of the triplet at 3.45 (ketoester) to 3.51 (ketoacid) indicated that the reaction was complete. The reaction mixture was concentrated on a rotary evaporator to remove ethanol. The aqueous residue was cooled in an ice bath, diluted with hexanes (60 mL), and vigorously stirred during careful dropwise addition of conc. aqueous HCl solution (heat evolved). When the mixture attained pH ˜ 1, the phases were separated and the aqueous layer was extracted with more hexanes (2×20 mL). The combined organic extracts were washed with DI water (30 mL), passed over a plug of anhydrous Na₂SO₄, and concentrated on the rotary evaporator. An NMR spectrum of the crude product was recorded to ascertain the presence of the desired ketoacid. The flask containing the residue from the rotary evaporation was capped with a septum and thoroughly purged with argon (balloon; needle vent). The flask was heated with a heat gun (while still sealed under argon and vented with a needle) until uncomfortably hot to the touch (100-130° C.), whereupon decarboxylation started. Bubbling of the residue was noticeable as the decarboxylation reaction proceeded. After approximately 10 min, no further bubbling was evident. The flask was cooled to room temperature and the residue was again analyzed by ¹H NMR, which revealed it to be nearly pure ketone. If desired, the crude ketone may be purified by column chromatography (gradient 1→3% v/v ether in hexanes). The crude ketone, however, is most advantageously introduced directly to the next steps. ¹H NMR δ 5.32 (m, 8H), 2.74 (t, 4H), 2.35 (t, 4H), 2.02 (m, 8H), 1.55-1.20 (m, 28H), 0.87 (t, 6H). ¹³C NMR δ 210.9, 130.0, 129.8, 128.0, 127.8, 42.6, 31.4, 29.5, 29.24, 29.22, 29.1, 29.0, 27.0 (2 overlapping peaks), 25.5, 23.7, 22.5, 14.0. LRMS m/z 499 [M+H]⁺, 521 [M+Na]⁺.

### (c) Ketalization of the ketone: 2-(2,2-Di((8Z,11Z)-heptadeca-8,11-dien-1-yl)-1,3-dioxolan-4-yl)ethan-1-ol

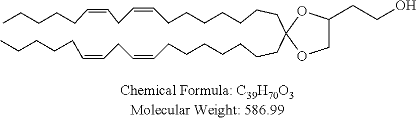 [SMILES: CCCCC/C=C\C/C=C\CCCCCCCC1(CCCCCCC/C=C\C/C=C\CCCCC)OCC(CCO)O1]

A mixture of ketone (1.0 mmol), 1,2,4-butanetriol (technical grade, ca. 90%, 236 mg, 2 mmol) and pyridinium p-toluenesulfonate (50 mg, 0.2 mmol) in 50 mL of toluene was refluxed under nitrogen overnight with continuous removal of water (Dean-Stark trap). Upon completion of the reaction, the mixture was cooled to room temperature, washed with water (2×30 mL), dried by passing over a plug of anhydrous Na₂SO₄ and evaporated. The yellowish oily residue (0.6 g) was purified by silica gel (230-400 mesh, 50 g) column chromatography, with dichloromethane as eluent, to afford 0.87-0.93 mmol (87-93%) of pure ketal. 1H NMR δ 5.36 (m, 8H), 4.24 (m, 1H), 4.09 (m, 1H), 3.81 (m, 2H), 3.53 (t, 1H), 2.78 (t, 4H), 2.21 (t, 1H [OH]), 2.05 (m, 8H), 1.82 (m, 2H), 1.65-1.53 (m, 4H), 1.42-1.23 (m, 32H), 0.89 (t, 6H). ¹³C NMR δ 130.2, 130.1, 128.0, 127.9, 112.6, 75.5, 69.9, 60.9, 37.8, 37.3, 35.4, 31.5, 29.9, 29.7, 29.5, 29.34, 29.27, 27.22, 27.19, 25.6, 24.0, 23.8, 22.6, 14.1. LRMS: m/z 587 [M+H]⁺, 609 [M+Na]⁺.

### (d) Ketal-alcohol mesylation: 2-(2,2-di((8Z,11Z)-Heptadeca-8,11-dien-1-yl)-1,3-dioxolan-4-ethyl methanesulfonate

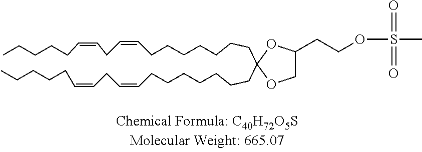 [SMILES: CCCCC/C=C\C/C=C\CCCCCCCC1(CCCCCCC/C=C\C/C=C\CCCCC)OCC(CCOS(C)(=O)=O)O1]

Neat Molecular Weight: 665.07 methanesulfonyl anhydride (290 mg, 1.6 mmol) was added to a solution of a ketal alcohol (prepared according to the previous section; 0.8 mmol) and dry triethylamine (242 mg, 330 uL, 2.4 mmol) in 5 mL of dry CH₂Cl₂. The resulting mixture was stirred at room temperature overnight. The mixture was diluted with 25 mL of CH₂Cl₂. the organic phase was washed with water (2×30 mL), passed over a plug of anhydrous Na₂SO₄, and evaporated to afford 510 mg of mesylate as yellowish oil. The crude mesylate was used in the following step without further purification. ¹H NMR δ 5.34 (m, 8H), 4.35 (m, 2H), 4.18 (m, 1H), 4.08 (m, 1H), 3.52 (t, 1H), 3.01 (s, 3H), 2.76 (t, 4H), 2.01 (m, 10H), 1.58-1.20 (m, 36H), 0.88 (t, 6H). ¹³C NMR δ 130.1, 130.0, 127.9, 127.8, 112.6, 72.2, 69.5, 67.1, 37.6, 33.3, 31.5, 31.4, 29.83, 29.81, 29.6, 29.5, 29.3, 29.21, 29.19, 27.2, 27.1, 25.6, 24.0, 23.7, 22.5, 14.0. LRMS: m/z 665 [M+H]⁺, 687 [M+Na]⁺.

### (e) Dimethylaminolysis of the mesylate to produce nor-KC2 ionizable lipid: 2-(2,2-Di((8Z,11Z)-heptadeca-8,11-dien-1-yl)-1,3-dioxolan-4-yl)-N,N-dimethylethan-1-amine

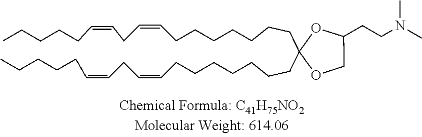 [SMILES: CCCCC/C=C\C/C=C\CCCCCCCC1(CCCCCCC/C=C\C/C=C\CCCCC)OCC(CCN(C)C)O1]

The above crude mesylate (500 mg) was added to 20 mL of a commercial 2.0 M solution of dimethylamine in THF. The resulting mixture was stirred at room temperature for 6 days, whereupon no more mesylate was apparent by TLC and/or ¹H NMR. Evaporation of the volatiles returned an oily residue that was purified by column chromatography on silica gel (230-400 mesh, 50 g) with 0-5% methanol gradient in dichloromethane as eluent, resulting in recovery of 350-400 mg of pure product. 1H NMR δ 5.36 (m, 8), 4.07 (m, 2H), 3.49 (t, 1H), 2.78 (t, 4H), 2.46-2.24 (m, 2H), 2.23 (s, 6H), 2.06 (m, 8H), 1.89-1.59 (m, 2H), 1.58 (m, 4H), 1.42-1.20 (m, 32H), 0.90 (br t, 6H). ¹³C NMR δ 130.1 (2 signals), 127.9 (2 signals), 112.1, 74.7, 69.9, 56.3, 45.4, 37.8, 37.5, 31.8, 31.5, 29.9 (2 signals), 29.7, 29.6 (2 signals), 29.5 (2 signals), 29.3 (2 signals), 27.2 (2 signals), 25.6, 24.0, 23.7, 22.6, 14.1. LRMS: m/z 614 [M+H]⁺

### Example 1: mRNA-Containing LNPs Comprising Nor-KC2 Ionizable Lipid Exhibit Transfection Efficiencies that are Superior to the MC3 Benchmark

LNP formulations containing the ionizable lipids, nor-KC2 or MC3, DSPC, cholesterol, and PEG-DMG were prepared containing mRNA encoding luciferase. The lipid nanoparticles comprise 50/10/38.5/1.5 mol % of ionizable lipid/DSPC/chol/PEG-DMG and the nitrogen-to-phosphorus ratio (N/P) was 6.

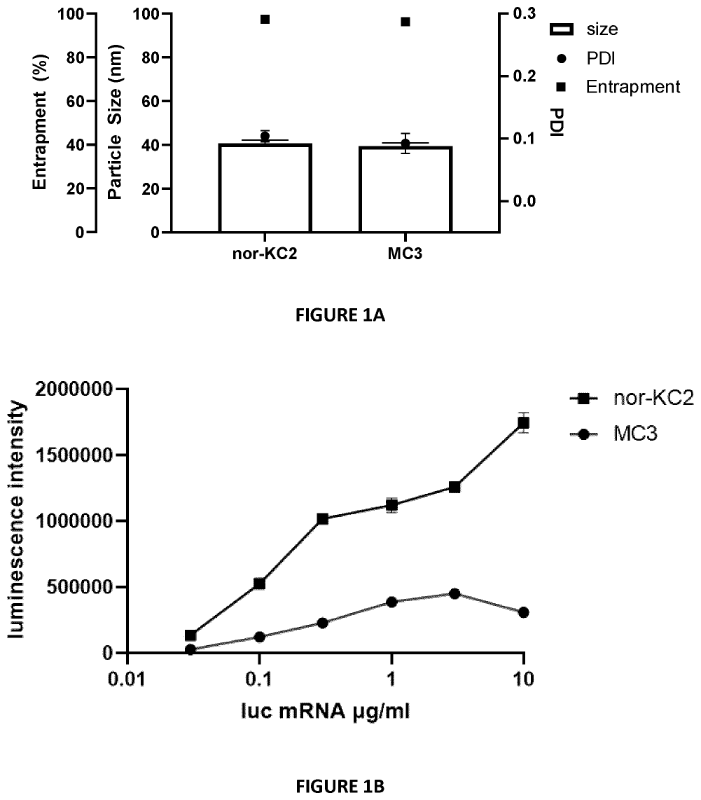

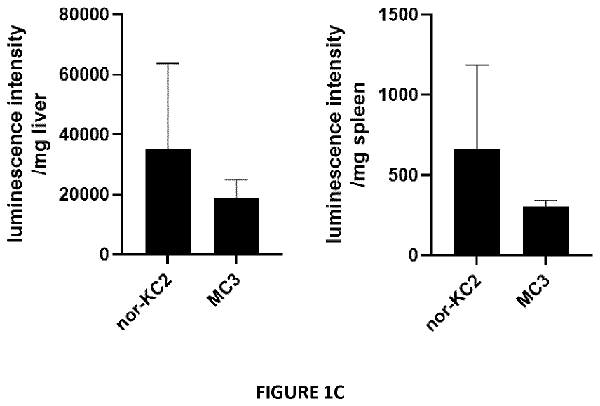

Transfection efficiencies of the LNPs comprising nor-KC2 verses the MC3 benchmark ionizable lipid are shown in FIG. 1B. The transfection efficiency of LNPs comprising nor-KC2 was superior to that of the MC3 benchmark lipid at each concentration of mRNA measured (μg/mL mRNA) after addition to Huh7. These results are particularly surprising since MC3 is a state-of-the-art ionizable lipid for LNP formulations encapsulating nucleic acid and has been reported as being more than 3 times more efficacious than KC2 (Jayaraman et al., 2012, Angew. Chem. Int. Ed., 51:8529-8533).

The results of characterization studies of LNP size, PDI and encapsulation efficiency of mRNA LNPs containing nor-KC2 and MC3 are shown in FIG. 1A. Advantageously, the mRNA-LNP containing nor-KC2 lipid exhibited similar size, PDI and encapsulation efficiency (entrapment) of the mRNA as the mRNA-LNP containing MC3.

### Example 2: mRNA-Containing LNPs Comprising Nor-KC2 Exhibit In Vivo Delivery of the mRNA to Liver and Spleen that is Superior to the MC3 Benchmark

LNP formulations containing 50/10/38.5/1.5 mol % of nor-KC2 or MC3 ionizable lipid/DSPC/chol/PEG-DMG and mRNA encoding luciferase were tested for in vivo biodistribution in the liver and spleen after injection to C57BL/6 mice. The mRNA dose was 1 mg/kg. Luminescence intensity in the liver or spleen was measured at 4 hours post-injection.

The results shown in FIG. 1C show that luminescence intensity per mg liver was higher for nor-KC2 than the MC3 benchmark. Similar results were found for luminescence intensity per mg spleen. The results showing superior delivery of mRNA to not only the liver, but also the spleen with nor-KC2-LNPs, are particularly surprising as MC3 is considered a more efficacious ionizable lipid than KC2.

### Example 3: siRNA-Containing LNPs Containing Nor-KC2 Exhibit Transfection Efficiencies that are Comparable to the MC3 Benchmark

As demonstrated in Example 1 and Example 2, mRNA encapsulating LNP formulations with nor-KC2 have superior transfection efficiency and significant improvements in biodistribution in vivo than MC3 formulations. In vitro physical characterization and transfection studies comparing nor-KC2 to MC3 were also conducted with LNPs containing siRNA. The results are discussed below.

LNP formulations containing the ionizable lipids, nor-KC2 or MC3, DSPC, cholesterol, and PEG-DMG were prepared containing siRNA encoding luciferase. The lipid nanoparticles comprise 50/10/38.5/1.5 mol % of ionizable lipid/DSPC/chol/PEG-DMG and the nitrogen-to-phosphorus ratio (N/P) was 3.

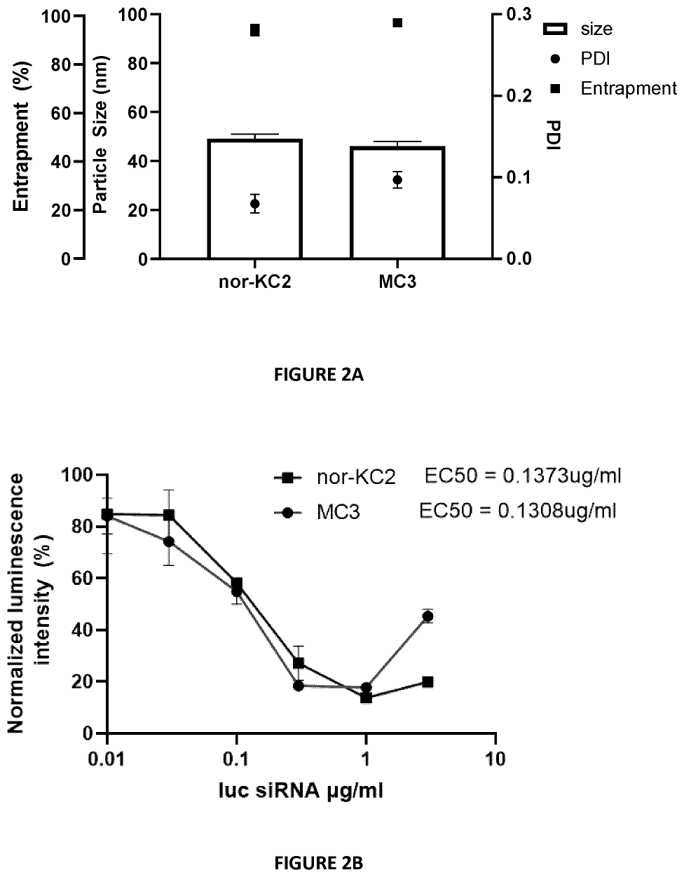

Transfection efficiency is shown in FIG. 2B. The transfection efficiency of siRNA LNPs containing nor-KC2 was comparable to that of the MC3 benchmark lipid, and was substantially better than MC3 at the highest dose tested (10 μg/mL siRNA). The half maximal effective siRNA concentration (EC₅₀) for LNP formulations of nor-KC2 and MC3 were comparable (nor-KC2 EC₅₀=0.1373 and MC3 EC₅₀=0.1308 μg/mL siRNA). These results are also surprising since KC2 lipid is considered less potent at siRNA transfection and thus would require more higher amounts of ionizable lipid to attain the same results as MC3.

The results of nor-KC2 vs. MC3 on LNP size, PDI and encapsulation efficiency of siRNA are shown in FIG. 2A. Advantageously, the LNP comprising nor-KC2 lipid exhibited similar size, PDI and encapsulation efficiency (entrapment) of the siRNA as MC3.

The examples are intended to illustrate the preparation of KC2-type lipids, formulations and properties thereof but are in no way intended to limit the scope of the invention.

1. A lipid having the structure of Formula D: 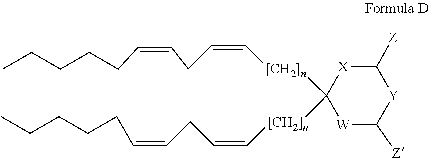
or a pharmaceutically acceptable salt thereof;
wherein n is 2 to 7;
W and X are independently O or S;
Y is absent or is present, wherein if:
Y is absent, then the carbon atoms to which Z and Z′ are respectively bound are directly linked by a C—C bond, or
Y is present, then Y is a bridge selected from a metheno (CH₂) bridge, etheno (CH₂CH₂) bridge and ethyno (CH═CH) bridge, the bridge optionally substituted with an alkylamino chain as defined by [—(CH₂)ₘ—NG¹G²G³],
wherein m is 1-5 and G¹ and G² are, independently, a C₁ to C₃ alkyl, G³ is absent, a hydrogen or a C₁ to C₃ alkyl,
or wherein NG¹G²G³ is a nitrogen heterocycle; and
Z and Z′ are, independently, H, or the alkylamino chain as defined by [—(CH₂)ₘ—NG¹G²G³].

2. The lipid of claim 1, wherein the C₁ to C₃ alkyl group is selected from methyl, ethyl, propyl and isopropyl.

3. The lipid of claim 1, wherein NG¹G²G³ is the nitrogen heterocycle and wherein the nitrogen heterocycle is 1-azetidinyl, 1-pyrrolidinyl, 1-piperidinyl, 1-azepanyl, 1-morpholinyl, 1-thiomorpholinyl or 1-piperazinyl.

4. The lipid of claim 1, wherein W and X are both oxygen atoms.

5. The lipid of claim 1, wherein n is 4 to 7.

6. The lipid of claim 5, wherein n is 7.

7. The lipid of claim 1, having the structure of Formula F: 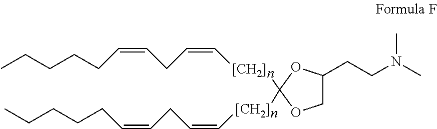
wherein n is 2 to 7.

8. The lipid of claim 7 having the structure of nor-KC2:

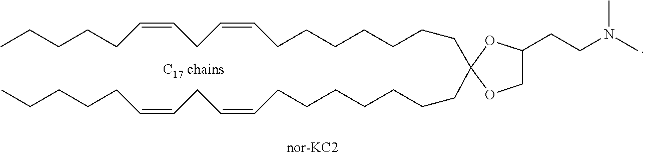 [SMILES: CCCCC/C=C\C/C=C\CCCCCCCC1(CCCCCCC/C=C\C/C=C\CCCCC)OCC(CCN(C)C)O1]

9. A drug delivery vehicle comprising the lipid of claim 1.

10. The drug delivery vehicle of claim 9, wherein the drug delivery vehicle is a lipid nanoparticle.

11. The drug delivery vehicle of claim 10, wherein the lipid nanoparticle includes a helper lipid selected from a sterol, a diacylglycerol, a ceramide or derivatives thereof.

12. The drug delivery vehicle of claim 11, wherein the diacylglycerol is selected from dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC) and mixtures thereof.

13. A method of producing the nor-KC2 lipid of claim 8 comprising:
(i) reacting methyl linoleate in a Claisen condensation reaction in the presence of a catalyst to produce a ketoester of the formula methyl (11Z,14Z)-2-((7Z,10Z)-hexadeca-7,10-dien-1-yl)-3-oxoicosa-11,14-dienoate;
(ii) reacting the ketoester via a hydrolysis and decarboxylation to produce a ketone of the formula (6Z,9Z,26Z,29Z)-Pentatriaconta-6,9,26,29-tetraen-18-one; and
(iii) preparing the nor-KC2 lipid from the ketone thereof using one or more synthesis steps resulting in an addition of an ionizable head group moiety to the ketone, thereby producing the nor-KC2 lipid.
